(19) United States
(12) Patent Application Publication
(10) Pub. No.: US 2014/0296303 A1
(43) Pub. Date: Oct. 2, 2014

(54) Use of Pyridoxamine to Treat and/or Prevent Disease Processes

(71) Applicant: Vanderbilt University, Nashville, TN (US)

(72) Inventors: Billy G. Hudson, Brentwood, TN (US); Paul Vosiyan, Nolensville, TN (US); Alp Ikizler, Nashville, TN (US)

(21) Appl. No.: 14/302,968

(22) Filed: Jun. 12, 2014

Related U.S. Application Data
(63) Continuation of application No. 13/659,371, filed on Oct. 24, 2012.
Application No. 13/659,371 is a continuation of application No. 12/775,641, filed on May 7, 2010, now abandoned.
Application No. 12/775,641 is a continuation of application No. 12/040,627, filed on Feb. 29, 2008, now abandoned.
Application No. 12/040,627 is a continuation of application No. 11/590,679, filed on Oct. 31, 2006, now abandoned.
(60) Provisional application No. 60/731,745, filed on Oct. 31, 2005.

(51) Int. Cl. A61K 31/44; A61K 9/00

(52) U.S. Cl. CPC A61K 31/44; A61K 9/0019; A61K 9/0053
USPC 514/351; 546/300

(57) ABSTRACT

Methods for treating and/or preventing at least one symptom of a disorder associated with oxidative stress, carbonyl stress, or combinations thereof in a subject. In some embodiments, the methods include administering to the subject an effective amount of pyridoxamine, an analog or derivative thereof, or a pharmaceutically acceptable salt of any of the foregoing. Also provided are methods for treating or preventing a nephropathy, acute renal injury, acute renal failure, or combinations thereof in a subject, and formulations adapted for intravenous administration comprising pyridoxamine, an analog or derivative thereof, or a pharmaceutically acceptable salt of any of the foregoing.

### CROSS REFERENCE TO RELATED APPLICATIONS

This application is based on and claims priority to U.S. Provisional Patent application Ser. No. 60/731,745, filed Oct. 31, 2005, herein incorporated by reference in its entirety.

### GRANT STATEMENT

This work was supported by grant R41-DK60251 from the U.S. National Institutes of Health. Thus, the U.S. government has certain rights in the presently disclosed subject matter.

### TECHNICAL FIELD

The presently disclosed subject matter relates to methods for treating and/or preventing a disorder of the kidney that promotes and/or is associated with oxidative and/or carbonyl stress in a subject. In particular, the presently disclosed subject matter relates to treating such subjects with an effective amount of pyridoxamine, or an analog or derivative thereof, or a pharmaceutically acceptable salt of any of the foregoing.

### BACKGROUND

Acute renal failure (ARF) is characterized by an abrupt loss of kidney function resulting in the failure to excrete urea and other nitrogenous waste products. A variety of conditions can produce ARF, including but not limited to low blood pressure, severe infections, and exposure to a variety of toxic substances. The Incidence of ARF in hospitalized patients varies from about 1% to about 15%, depending upon the population at risk and the specific criteria used for defining renal failure, with an even higher incidence in critically II patients.

Often, sepsis is complicated by ARF and, conversely, ARF is precipitated by and/or complicated by sepsis. Sepsis is the cause of ARF in approximately 50% of the patients in intensive care units (ICUs). Radiographic contrast agents are one of the leading causes of hospital-acquired ARF.

No established pharmacologic therapy is currently available for the prevention or treatment of ARF. Its management is primarily supportive, with renal replacement therapy (e.g., dialysis) serving as the cornerstone in patients with severe ARF. The mortality rates associated with ARF have remained very high (in excess of 50%) over the past 50 years despite advances in the use of hemodialysis and other renal replacement therapies. Although ARF is associated with a high mortality rate, patients who survive have an excellent prognosis with regard to the eventual return of renal function. In about 90% of patients who survive, restoration of renal function permits discontinuation of dialysis.

To date, intervention studies in large, randomized clinical trials have failed to show significance in preventing acute renal failure in critically ill patients. Thus, there is a long-standing and continuing need for new treatments for ARF. This and other needs are addressed by the methods of the presently disclosed subject matter.

### SUMMARY

This Summary lists several embodiments of the presently disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

The presently disclosed subject matter provides methods for ameliorating at least one symptom of a disorder, such as a kidney disorder, associated with oxidative stress, carbonyl stress, or combinations thereof in a subject. In some embodiments, the methods comprise administering to the subject an effective amount of pyridoxamine (PM), or an analog or derivative thereof, or a pharmaceutically acceptable salt of any of the foregoing, whereby at least one symptom of the kidney disorder associated with oxidative stress, carbonyl stress, or combinations thereof in a subject is ameliorated. In some embodiments, the kidney disorder associated with oxidative stress, carbonyl stress, or combinations thereof in the subject results from a medical condition associated with elevated levels of reactive carbonyl species (RCS), reactive oxygen species (ROS), advanced glycation end products (AGE), or combinations thereof. In some embodiments, the kidney disorder associated with oxidative stress, carbonyl stress, or combinations thereof in the subject is selected from the group consisting of diabetic nephropathy, acute renal injury, acute renal failure, and combinations thereof. In some embodiments, the kidney disorder associated with oxidative stress, carbonyl stress, or combinations thereof in the subject is selected from the group consisting of acute kidney injury and acute kidney failure. In some embodiments, the administering reduces formation of, reactivity of, or both formation of and reactivity of at least one RCS, ROS, of AGE.

The presently disclosed subject matter also provides methods for treating or preventing a nephropathy in a subject. In some embodiments, the methods comprise administering to the subject an effective amount of pyridoxamine, or an analog or derivative thereof, or a pharmaceutically acceptable salt of any of the foregoing, wherein the effective amount comprises an amount sufficient to ameliorate the nephropathy in the subject, to prevent or delay onset of the nephropathy in the subject, or combinations thereof. In some embodiments, the nephropathy is selected from the group consisting of acute kidney injury and acute kidney failure. In some embodiments, the nephropathy is associated with exposure to a radiographic contrast agent, sepsis, chemotherapy, congestive heart failure, cardiovascular disease, or combinations thereof.

The presently disclosed subject matter also provides methods for treating or preventing acute renal injury, acute renal failure, or combinations thereof in a subject. In some embodiments, the methods comprise administering to the subject an effective amount of pyridoxamine, or an analog or derivative thereof, or a pharmaceutically acceptable salt of any of the foregoing. In some embodiments, the acute renal injury, acute renal failure, or combinations thereof is associated with exposure to a radiographic contrast agent, sepsis, chemotherapy, congestive heart failure, cardiovascular disease, or combinations thereof. In some embodiments, the amount administered to the subject is an amount sufficient to increase the time required for at least one symptom associated with the acute renal failure to develop, to reduce the severity of at least one symptom associated with the acute renal failure, to reduce the time that at least one symptom associated with the acute renal failure is present within the subject, and combinations thereof.

In some embodiments of the disclosed methods, the administering is by a route selected from the group consisting of intravenous administration, parenteral administration, and oral administration.

In some embodiments of the disclosed methods, the effective amount is selected from the group consisting of less than about 1 mg/day, about 1-10 mg/day, about 10-50 mg/day, about 50-100 mg/day, about 100-200 mg/day, about 200-300 mg/day, about 300-400 mg/day, about 400-500 mg/day, and more than 500 mg/day.

The presently disclosed subject matter also provides formulations comprising pyridoxamine, or an analog or derivative thereof, or a pharmaceutically acceptable salt of any of the foregoing. In some embodiments, the formulation is adapted for intravenous administration into a subject. In some embodiments, the formulation is adapted for intravenous administration into a human. In some embodiments, the formulation comprises an amount of pyridoxamine, or the analog or derivative thereof, or the pharmaceutically acceptable salt of any of the foregoing, sufficient to ameliorate at least one symptom associated with acute renal failure in the subject, to prevent or delay onset of acute renal failure in the subject, or combinations thereof.

Accordingly, it is an object of the presently disclosed subject matter to provide new methods and compositions for use in treating and/or preventing a kidney disorder in a subject. This and other objects are achieved in whole or in part by the presently disclosed subject matter.

An object of the presently disclosed subject matter having been stated hereinabove, other objects will be evident as the description proceeds and as best described hereinbelow.

### BRIEF DESCRIPTION OF THE DRAWINGS

FIGS. 1A and 1B depict the inhibition of the formation of AGE Nε-carboxymethyllysine (CML) on BSA and on collagen IV by pyridoxamine (PM).

FIG. 1A is a graph depicting the conversion of Amadori intermediate to CML measured using anti-CML antibody. The data points on the graph correspond to the following: : no PM; ▴:15 mM PM; ▪:1 mM PM; ♦: 0.1 mM PM; and ▾: nonglycated BS.

FIG. 1B is a bar graph depicting the results of enzyme-linked immunosorbent assays (ELISAs) of collagen IV coated 96-well plates incubated in the presence of normal (5 mM) and diabetic (30 mM) concentrations of glucose with or without PM and developed with an anti-CML antibody. The secondary antibody used in the assay was an alkaline phosphatase conjugated goat anti-rabbit IgG from Sigma-Aldrich Co. (St. Louis, Mo., United States of America). Incubations were carried out in 200 mM sodium phosphate buffer, pH 7.5 at 37° C. for 40 days.

FIGS. 2A and 2B are graphs depicting the results of scavenging of reactive carbonyl species glyoxal (GO) and methylglyoxal (MGO) by PM. Carbonyl compounds GO (FIG. 2A) and MGO (FIG. 2B) were incubated with or without PM in 200 mM sodium phosphate buffer, pH 7.5 at 37° C. The residual free carbonyl groups were measured using Girard's reagent T. The data points on the graphs correspond to the following: : without PM; ▪: with PM.

FIGS. 3A-3C are graphs depicting the inhibition of accumulation of hydroxyl radical by PM. Hydroxyl radical, the most active reactive oxygen specie, was formed using three different reactions. Incubations were carried out with and without PM in 200 mM sodium phosphate buffer, pH 7.5 at 37° C. for 24 hours. Accumulation of hydroxyl radical was measured using specific oxidation of benzoic acid.

FIG. 3A is a graph depicting hydroxyl radical generation in the presence of various concentrations of PM.

FIG. 38 is a bar graph depicting hydroxyl radical generation from the Fenton reaction in the presence or absence of 20 mM PM.

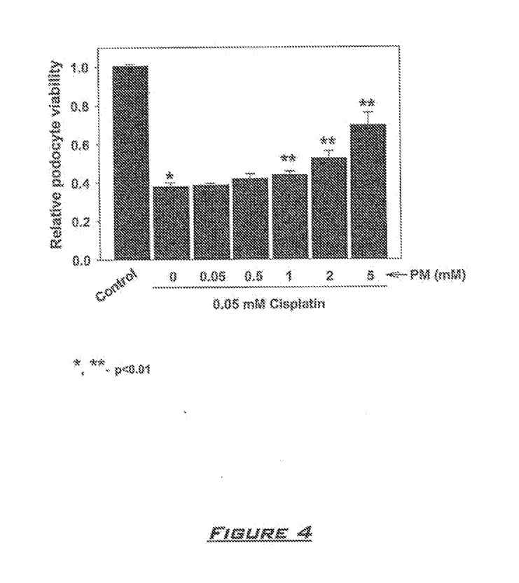

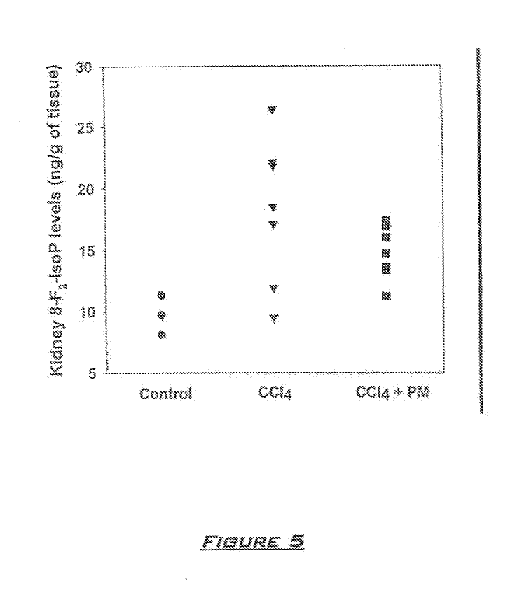

FIG. 3C is a bar graph depicting hydroxyl radical generation from a protein-Amadori intermediate (Am-BSA) or from BSA in the presence of 0 mM, 2 mM, or 20 mM PM.

FIG. 4 is a bar graph depicting the effects of different treatment amounts of PM on cisplatin-induced podocyte viability after 22 hours of treatment. The asterisks indicate conditions that where there was a statistically significant difference (p<0.01) between the relative podocyte viability and the negative control (i.e., no PM treatment; *) or between the negative control and individual treatment conditions (**).

FIG. 5 is a plot depicting the effect of PM administration on the levels of a marker of oxidative stress (8-F₂-IsoP) in the carbon tetrachloride (CCl₄) rat model disclosed herein as determined by gas-chromatography/mass-spectrometry (GC-MS). The data points on the plot correspond to the following: : no CCl₄ or PM; ▾: CCl₄ alone; and ▪: CCl₄ and PM.

FIGS. 6A and 6B are graphs depicting the effect of PM treatment on urinary oxalate excretion in animals with experimental hyperoxalura. Sprague-Dawley male rats (49-52 days old) were randomized on Day 1 to receive either ethylene glycol (0.75% v/v in drinking water (450 mg/day/kg body wt.); EG group) or no treatment (Control group). After Day 14, animals within each group were pair-matched according to their oxalate level. One member of each pair was then randomly assigned to receive PM (3 mg/ml or 180 mg/day/kg body wt.) either in drinking water (PM group) or in 0.75% ethylene glycol (EG+PM group). Urine samples were collected under toluene (to inhibit bacteria growth) in 50 ml tubes with hydrochloric acid to minimize spontaneous breakdown of urinary ascorbic acid to oxalate. Urinary oxalate was measured as described under Methods. Each symbol represents mean value±S.E. (n=5); at the time points indicated by asterisks, differences between the EG group and the EG+PM group were statistically significant (p<0.05). The data points on the graphs correspond to the following: —Control group; ▪—EG group; —PM group; ⋄—EG+PM group.

In FIG. 6A, the results of PM given continuously starting from Day 14 are presented.

In FIG. 6B, the results for a separate experiment in which PM treatment was started on Day 14 and was discontinued on Day 28 are presented. In this experiment, the elevated levels of oxalate excretion in Control group on Day 3 are most likely related to animal adaptation. Note that the oxalate levels went down and leveled off before the beginning of PM treatment.

### DETAILED DESCRIPTION

The presently disclosed subject matter provides methods for treating and/or preventing a disorder, such as but not limited to a kidney disorder, that is associated with oxidative stress, carbonyl stress, or combinations thereof. In some embodiments, the disorder can be mediated at least in part by elevated levels of reactive carbonyl species (RCS), reactive oxygen species (ROS), and/or advanced glycation end products (AGE) in a subject. In some embodiments, the methods comprise administering to the subject an effective amount of pyridoxamine, or an analog or derivative thereof, or a pharmaceutically acceptable salt of any of the foregoing. For example the effective amount can be an amount sufficient to reduce formation and/or reactivity of at least one RCS, ROS, or AGE. In some representative embodiments the disorder comprises acute renal injury and/or acute renal failure.

### I. DEFINITIONS

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter.

Following long-standing patent law convention, the terms “a”, “an”, and “the” mean “one or more” when used in this application, including the claims. Thus, for example, the phrase “a reactive oxygen species” refers to one or more reactive oxygen species.

The term “about”, as used herein when referring to a measurable value such as an amount of weight, time, dose (e.g., a dose of PM), etc., is meant to encompass variations of in some embodiments ±20%, in some embodiments ±10%, in some embodiments ±5%, in some embodiments ±1%, and in some embodiments ±0.1% from the specified amount, as such variations are appropriate to perform the disclosed methods.

As used herein, the term “and/or” refers to alternatives in which one or more of the listed entities is present. For example, the phrase “A and/or B” refers to alternatives wherein A is present, B is present, or both A and B are present. In those cases where more than two alternatives are present, the phrase “and/or” refers to alternatives in which any one of the listed entities is present, all of the listed entities are present, or any subset of listed entities is present.

As used herein, the phrase “associated with” refers to a relationship between two or more occurrences that one of ordinary skill in the art would recognize is normally or frequently observable when one or more of the occurrences is present. For example, a “symptom associated with a disorder in a subject” Is a symptom that is normally, frequently, or sometimes present in the subject when the subject has the disorder. It is understood, however, that the symptom need not necessarily be indicative of the disorder, causative of the disorder, or absent in the subject in the absence of the disorder. Thus, the phrase “associated with” does not necessarily imply a causal relationship between the two or more occurrences, although in some embodiments a causal relationship can exist.

For example, in some embodiments the phrase “kidney disorder that is associated with oxidative stress, carbonyl stress, or combinations thereof” refers to any nephropathy at least one symptom of which is caused by or modulated by oxidative stress, carbonyl stress, or combinations thereof, as those terms would be understood by one of ordinary skill in the art after review of the instant disclosure. In some embodiments, a “kidney disorder that is associated with oxidative stress, carbonyl stress, or combinations thereof” is a medical condition associated with elevated levels of reactive carbonyl species (RCS), reactive oxygen species (ROS), and/or advanced glycation end products (AGE). In some embodiments, a “kidney disorder that is associated with oxidative stress, carbonyl stress, or combinations thereof” comprises acute renal injury (ARI), acute renal failure (ARF), and combinations thereof.

As used herein with respect to compositions comprising pyridoxamine (PM) or an analog or derivative thereof, the phrase “effective amount of pyridoxamine (PM), or an analog or derivative thereof”, refers to an amount of PM or an analog or derivative thereof that when administered to a subject as a single dose or in multiple doses leads to an amelioration of (e.g., an improvement of, a decreased duration of, etc.) at least one symptom of a disorder disclosed herein. In some embodiments, the disorder and/or the symptom is associated with oxidative stress, carbonyl stress, or combinations thereof in the subject. In some embodiments, the effective amount reduces formation of, reactivity of, or both formation and reactivity of at least one RCS, ROS, or AGE in order to ameliorate at least one symptom of the disease associated with oxidative stress, carbonyl stress, or combinations thereof in the subject.

### II. OXIDATIVE STRESS AND CARBONYL; STRESS GENERALLY

Oxidative stress is the term used to describe a physiological state that can promote and/or can be associated with an increase in the level of reactive oxygen species (ROS) and reactive nitrogen species (NOS), either from injury or disease processes, or a decrease in endogenous protective anti-oxidative capacity, or both. Oxidative stress is usually accompanied by carbonyl stress characterized by an increase in production of low molecular weight reactive carbonyl species (RCS). In many types of illnesses, including but not limited to sepsis, trauma, burn injury, acute pancreatitis, liver injury, severe diabetes, acute respiratory distress syndrome, AIDS, and acute renal failure, increased oxidative stress and/or carbonyl stress can occur.

II.A. Reactive Oxygen Species

The two free electrons of the diatomic oxygen molecule provide for a number of activated electronic states that can result in the formation of a number of reactive oxygen molecules such as hydrogen peroxide, hydroxyl radical, superoxide radical, peroxyl and alkoxyl radicals, and others, collectively referred to as reactive oxygen species (ROS). ROS are formed in different biological reactions such as glycoxidation and lipid peroxidation and are mediators of a number of physiological processes. These species are powerful messenger molecules involved in gene regulation, thereby resulting in, for example, the synthesis of cytokines and/or adhesion molecules necessary for defending inflammatory processes. Because of their high reactivity, their function as mediators of biological activity are likely localized to specific tissue compartments. Endogenous enzymes exist to control physiological levels of ROS, such as superoxide dismutase (superoxide) and catalase (hydrogen peroxide).

The study of ROS in normal physiological processes, as well as their pathogenic effects when present in excess levels, has been a challenging area of investigation because of their high reactivities toward biological molecules and pathways. However, advances in analytical methods can now identify and quantify compounds in complex biological systems that are reaction products of ROS. These new methodologies have provided the opportunity to study ROS formation and reactivity in a number of in vitro, ex vivo, and animal model systems, which has further defined the role of these reactive molecules in health and disease. In addition, ROS formation and reactivity has been applied to clinical studies, and measurements have been correlated with disease progression and therapeutic response.

Elevated levels of ROS can be pathogenic. They can promote unwanted signaling and gene expression, and they form chemical adducts with biomolecules that can effect many physiological processes. The fact that nature has apparently evolved specific mechanisms to control their levels and distribution (e.g., superoxide dismutase and catalase) speaks to the Importance of maintaining localized concentrations of ROS at specific physiological levels.

II.B. Reactive Carbonyl Species

Reactive carbonyl species (RCS), including but not limited to glyoxal (GO), methylglyoxal (MGO), and glycolaldehyde (GLA), can be generated by autoxidation of glucose or Schiff base intermediate formed during the reaction of glucose with protein amino groups. A protein-Amadori adduct, another glycation intermediate, is a major source of another carbonyl compound, 3-deoxyglycosone (3-DG). MGO can also originate from either spontaneous or enzymatic degradation of triose phosphates derived from glucose. Besides glycoxidation reactions, reactive carbonyl species can also derive from other sources such as ascorbate, polyunsaturated lipids, and amino acids. RCS can damage proteins and DNA by reacting with nudeophilic groups of biological macromolecules. These small carbonyl compounds diffuse easily from the sites of formation to the Interior regions of macromolecules, thus further propagating the damage.

### III. OXIDATIVE AND CARBONYL STRESS IN CRITICALLY ILL PATIENTS

Oxidative stress and increased levels of ROS are characteristics of sepsis, trauma, cardiogenic shock, burn injury, diaphragm fatigue, acute pancreatitis, liver injury, severe diabetes, ischemia-reperfusion injury, acute renal injury, acute renal failure, acute respiratory distress syndrome (ARDS), systemic inflammatory response syndrome, multiple organ dysfunction, and AIDS. Many of these conditions result in the induction and/or aberrant regulation of the immuno-inflammatory system, which results in overproduction and/or insufficient elimination of ROS.

For example, hundreds of thousands of sepsis patients are treated in intensive care units around the world every year, and sepsis is characterized by aberrant regulation of the immuno-inflammatory system (see Macdonald et al. (2003) 90 Br J Anaesth 90:221-32). In sepsis, potential sources of ROS include the mitochondrial electron transport chain, ischemia-reperfusion induced xanthine oxidase activity, neutrophil activation, and arachidonic acid metabolism. Activated neutrophils, for example, produce superoxide anion as a part of their function as phagocytes, and also produce the free radical nitric oxide (NO⁻), which can react with superoxide to produce peroxynitrite. Peroxynitrite can decompose to form the hydroxyl radical (OH⁻). Additionally, ischemia-reperfusion injury results in the induction of the enzyme xanthine oxidase, which produces superoxide in a side reaction. Superoxide results in the recruitment and activation of neutrophils and their adherence to endothelial cells, which stimulates the formation of xanthine oxidase in the endothelium, with leads to further superoxide production.

Thus, the induction of an immune response or inflammation results in the production of ROS, and when the innate antioxidant system of the patient is compromised, such as in cases of critical illness, the homeostatic balance between oxidants and antioxidants is tipped unfavorably towards the overproduction and/or insufficient elimination of ROS and other oxidants.

In the case of acute renal injury and/or acute renal failure, ROS and RCS are factors contributing to the onset of this syndrome.

In addition, recent studies have determined that many of the disease processes and syndromes that exhibit oxidative and/or carbonyl stress also exhibit elevated AGE levels, which could be contributing to pathology and patient outcome.

Several other conditions typically experienced by the critically ill patient have been associated with oxidative and/or carbonyl stress. For example, ischemia-reperfusion injury results in the production of ROS when the oxygen in the restored blood flow forms free radicals within cells that can damage cellular proteins, DNA, and the plasma membrane. In response to ischemia, endothelial cells induce the production of xanthine oxidase, which produces ROS (e.g., O₂⁻).

Additionally, some of the treatments and/or diagnostic procedures that a critically ill patient might be expected to encounter in the ICU have also been associated with the production of ROS, RCS, and/or AGEs. For example, many critically ill patients receive some form of radiographic assessment, and radiographic contrast agents are known to cause nephrotoxicity. The cause of this toxicity is hypothesized to involve ROS (see Tepel at al. (2000) 343 N Engl J Med 180-4). Additionally, critically ill patients often require respiratory support using a mechanical ventilator, and when such patients are weaned from ventilators, they commonly experience diaphragm fatigue associated with the production of increased ROS levels as the muscles of the diaphragm resume functioning.

Thus, for the critically ill patient, oxidative and/or carbonyl stress can contribute to increased mortality and/or lower quality of life. The lipid peroxidation, protein oxidation, and mutations in mitochondrial DNA that can result from oxidative stress and/or carbonyl stress can result in cell death that contributes to the disease processes experienced by the critically ill patient. For this patient population in particular, bodily antioxidant defense systems can become overwhelmed, resulting in increases in ROS, RCS, and AGE levels and exacerbating the patient's condition.

Accordingly, treatment of ARF and/or ARI includes consideration of several stages of ARF and/or ARI disease and outcomes including, but not limited to the development of ARF and/or ARI from a number of conditions that often lead to ARF and/or ARI (e.g., contrast dye; sepsis; chemotherapy; congestive heart failure; advanced cardiovascular disease; etc.); and the progression of ARF to multi-organ failure and/or the development of a systemic inflammatory response that leads to death. Consequently, the emergence of oxidative stress conditions that promote the onset of ARF and/or ARI, the emergence of these conditions to patients in the intensive care unit (ICU), the severity of ARF and/or ARI that develops from these conditions, the degree of restoration of renal function that results from treatment, and/or the survival rate of patients that receive treatment can be considered in the context of and within the scope of the presently disclosed subject matter.

### IV. METHODS OF TREATMENT AND/OR PREVENTION

The presently disclosed subject matter provides in some embodiments methods for treating and/or preventing a disease process that promotes and/or is associated with oxidative and/or carbonyl stress. In some embodiments, the disease process is mediated at least in part by elevated levels of reactive carbonyl species (RCS), reactive oxygen species (ROS), and/or advanced glycation end products (AGE) in a subject. In some embodiments, the methods comprise administering to the subject an effective amount of pyridoxamine, or an analog or derivative thereof, or a pharmaceutically acceptable salt of any of the foregoing. In some embodiments, the administering reduces formation and/or reactivity of at least one RCS, ROS, or AGE to thereby treat and/or prevent the disease process in the subject.

As used herein, in some embodiments, any disease process that promotes and/or is associated with oxidative and/or carbonyl stress can be treated, including but not limited to those mediated at least in part by elevated levels of reactive carbonyl species (RCS), reactive oxygen species (ROS), and/or advanced glycation end products (AGE). In some embodiments, the term “disease process” can refer to any disease at least one symptom of which results from elevated levels of RCS, TOS, and/or AGE. Exemplary disease processes include, but are not limited to sepsis, trauma, burn injury, acute pancreatitis, liver injury, severe diabetes, acute respiratory distress syndrome, and AIDS. Thus, the methods disclosed herein can be used to treat and/or prevent the development and/or progression of these disease processes, among others. In some embodiments, the disease process can primarily be mediated by the elevated levels of RCS, ROS, AGE, and combinations thereof.

The presently disclosed subject matter also provides methods for treating and/or preventing a disorder, such as but not limited to a kidney disorder, that is associated with oxidative stress, carbonyl stress, or combinations thereof. In some embodiments, the disorder can be mediated at least in part by elevated levels of reactive carbonyl species (RCS), reactive oxygen species (ROS), and/or advanced glycation end products (AGE) in a subject. In some embodiments, the methods comprise administering to the subject an effective amount of pyridoxamine, or an analog or derivative thereof, or a pharmaceutically acceptable salt of any of the foregoing. For example the effective amount can be an amount sufficient to reduce formation and/or reactivity of at least one RCS, ROS, or AGE. In some representative embodiments the disorder comprises acute renal injury and/or acute renal failure.

As used herein, the term “treatment”, and grammatical variants thereof, refers to a medical intervention that is designed to reduce or eliminate at least one symptom resulting from a disease process as described herein. The term “prevention”, and grammatical variants thereof, refers to a medical intervention that is designed to retard or prevent the initial development or subsequent progression of at least one symptom resulting from a disease process as described herein. Thus, in some embodiments “prevention” and “treatment” can overlap. As such, the terms are used substantially interchangeably herein, although it is understood that “treatment” implies that at least one symptom resulting from a disease process as disclosed herein has become manifest in some observable and/or quantifiable fashion.

In some embodiments, the methods disclosed herein provide for treatment and/or prevention of acute renal failure and/or acute renal injury in a subject. As used in this context, the term “prevent” is also intended to relate to a prophylactic approach, such that “preventing” includes both modulating the initial development of a disease process as well as modulating the further development of (i.e., the worsening of) a disease process. It is understood that the degree of prevention/prophylaxis need not be absolute (e.g., complete prevention of the development of a disease process such that the subject does not develop the disease process at all), and that intermediate levels of prevention/prophylaxis including, but not limited to increasing the time required for at least one symptom resulting from a disease process to develop, reducing the severity of at least one symptom resulting from a disease process, and reducing the time that at least one symptom resulting from a disease process is present within the subject are all examples of prevention/prophylaxis. With respect to the latter two circumstances, these are examples wherein “prevention/prophylaxis” and “treatment” can be considered to coincide.

It is also understood that the disclosed methods can be used as part of a combination therapy, and need not be employed as the sole therapy to address a disease process as disclosed herein. For example, PM administration can be combined with antibiotic therapy in septicemia, and PM administration can be combined with dialysis in ARF.

The methods disclosed herein can be used to provide a benefit to subjects suffering from disorders and/or disease processes described herein. As used herein, the phrase “provide a benefit” to the subject is intended to refer to a qualitative or quantitative benefit provided to the subjects on whom the instant methods are performed relative to similarly situated subjects (i.e., subjects at the same level of disease, with the same prognosis prior to treatment, etc.) that were not treated with PM administration.

In some embodiments, the benefit is an increase in survival. “Survival” is therefore intended to encompass absolute survival (e.g., the treatment results in the disease process being non-fatal in a subject in whom it would have been fatal in the absence of treatment) as well as improvements in the quality of life of the patient (e.g., the duration of the disease and/or a symptom of the disease is reduced, the severity of the disease and/or a symptom of the disease is reduced, and/or the ability to tolerate higher doses of other combination therapies, if used, is increased).

Thus, for example, “survival” can be expressed as a survival rate (i.e., the percentage of similarly situated subjects that survive for a particular time period). Non-limiting examples of survival rates include, but are not limited to 90-day survival rates, 120-day survival rates, and 1-year survival rates. In some embodiments, the survival rate relates to the time period a critically ill subject might stay in the ICU (e.g., 2 to 4 week survival rates).

### IV.A Subjects

The term “subject” as used herein refers to a member of any vertebrate species. The methods of the presently disclosed subject matter are particularly useful for warm-blooded vertebrates. Thus, the presently disclosed subject matter concerns mammals and birds. Provided herein is the treatment of mammals such as humans, as well as those mammals of importance due to being endangered (such as Siberian tigers), of economic importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), and horses. Also provided is the use of the disclosed methods on birds, including those kinds of birds that are endangered, kept in zoos, as well as fowl, and more particularly domesticated fowl including, but not limited to poultry, such as turkeys, chickens, ducks, geese, guinea fowl, and the like, as they are also of economic importance to humans. Thus, provided is the treatment of livestock, including but not limited to domesticated swine (pigs and hogs), ruminants, horses, poultry, and the like.

IV.B. Inhibitory Activity of Pyridoxamine Toward AGE, ROS, and RCS Formation

Pyridoxamine (PM) is a very potent inhibitor of advance glycation end product (AGE) formation. AGEs are an established pathogenic factor in diabetic renal disease and probably other diabetic complications. Additionally, pyridoxamine can inhibit other types of chemical processes associated with oxidative and carbonyl stress including the formation of ROS and RCS. Elevated levels of ROS and/or RCS are considered a pathogenic factor in a broad range of diseases and syndromes. Pyridoxamine can also inhibit the reactivity of ROS and RCS toward biological molecules, thus inhibiting the pathologic potential of the elevated levels of these species.

In addition to inhibiting the formation of advance glycation end products (AGEs), pyridoxamine inhibits the formation of ROS and RCS. ROS and RCS are normally present in tissues at relatively low levels. However, after injury and/or the onset of a wide range of disease processes, particularly those found in critically II patients, oxidative and/or carbonyl stress can develop and generate elevated pathogenic levels of ROS and RCS. For example, elevated levels of ROS, RCS, and AGEs have been found in ARF.

ARF outside the Intensive care unit (ICU) is relatively rare, and generally is not fatal. The acute renal failure that develops in the critically ill patient, which can be brought on by hypertension, congestive cardiac failure, diabetes, chronic infection, and drug therapies such as anti-cancer and anti-inflammatory therapies, has a mortality rate of 50 to 70 percent. It is believed that the further increase in oxidative and/or carbonyl stress that develops after the onset of ARF is a significant factor in this high mortality rate.

Thus, as disclosed herein, pyridoxamine inhibitory activity toward ROS, RCS, and AGEs, provides that this compound can impact both the onset of ARF as well as the high mortality rate experienced by critically ill ARF patients. While not wishing to be restricted to any particular theory of operation, the presently disclosed subject matter pertains at least in part to (1) the discovery of the inhibitory activity of pyridoxamine against the formation and reactivity of dicarbonyl compounds, “reactive oxygen species” (ROS), and “advanced glycation end-products” (AGEs) in the development of ARF; and (2) the pathogenic effect of these compounds in critically ill patients that develop ARF.

IV.C. Pyridoxamine, and Analogs and Derivatives Thereof

As used herein, the term “pyridoxamine” (PM) refers to 4-(aminomethyl)-5-(hydroxymethyl)-2-methyl-pyridin-3-ol, and has the structure shown in Structure A:

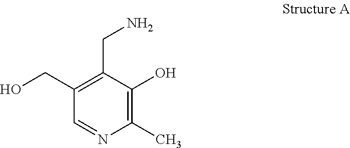

It is the 4-aminomethyl form of vitamin B6, and is also referred to as pyridoxylamine. As such, these terms are used interchangeably herein to refer to a compound of Structure A.

The presently disclosed methods can also employ analogs or derivatives of PM. By “analog or derivative” is intended a compound that contains a structure similar but not identical to Structure A, yet retains all or some of the biological activity of PM. An analog or derivative of PM retains in some embodiments at least 10%, in some embodiments at least 20%, in some embodiments at least 30%, in some embodiments at least 40%, in some embodiments at least 50%, in some embodiments at least 60%. In some embodiments at least 70%, in some embodiments at least 80%, in some embodiments at least 90%, in some embodiments at least 95% of the activity of PM in inhibiting the formation of ROS, RCS, or AGE in an in vivo or in vitro assay (e.g., one of the assays disclosed herein). In some embodiments, an analog or derivative has 100% or greater of the activity relative to PM in the same assay. Analogs and derivatives can be naturally occurring, or can be created synthetically in accordance with art-recognized techniques as would be apparent to one of ordinary skill in the art after a review of the present disclosure.

Exemplary analogs and derivatives have the structure presented in Structure B:

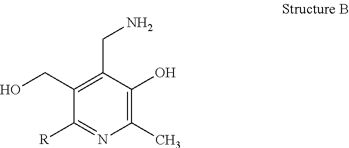

where R is selected from the group consisting of alkyl, branched alkyl, and substituted alkyl; aryl, and substituted aryl, alkoxy, branched alkoxy, and substituted alkoxy; benzyloxy; alkylcarboxylic acid, branched alkylcarboxylic acid, substituted alkylcarboxylic acid, amino, substituted amino, alkylamino, and dialkylamino.

A named R group will generally have the structure that is recognized in the art as corresponding to R groups having that name. For the purposes of illustration, representative R groups as enumerated above are defined herein. These definitions are intended to supplement and illustrate, not preclude, the definitions known to those of skill in the art.

As used herein, the term “alkyl” means C₁₋₁₂ inclusive (i.e., carbon chains comprising 1, 2, 3, 4, 5, 6, 7, 8, 9, 10, 11, or 12 carbon atoms; also, in some embodiments, C₁₋₆ inclusive) linear, branched, or cyclic, saturated or unsaturated (i.e., alkenyl and alkynyl)hydrocarbon chains, including for example, methyl, ethyl, propyl, isopropyl, butyl, isobutyl, tert-butyl, pentyl, hexyl, ethenyl, propenyl, butenyl, pentenyl, hexenyl, butadienyl, propynyl, butynyl, pentynyl, hexynyl, and allenyl groups.

The alkyl group can be optionally substituted with one or more alkyl group substituents which can be the same or different, where “alkyl group substituent” includes alkyl, halo, aryl, arylamino, acyl, hydroxy, aryloxy, alkoxyl, alkylthio, arylthio, aralkyloxy, aralkylthio, carboxy, alkoxycarbonyl, oxo, and cycloalkyl. In this case, the alkyl can be referred to as a “substituted alkyl”. Representative substituted alkyls include, for example, benzyl, trifluoromethyl, and the like. There can be optionally inserted along the alkyl chain one or more oxygen, sulfur or substituted or unsubstituted nitrogen atoms, wherein the nitrogen substituent is hydrogen, alkyl (also referred to herein as “alkylaminoalkyl”), or aryl. Thus, the term “alkyl” can also include esters and amides. “Branched” refers to an alkyl group in which an alkyl group, such as methyl, ethyl, or propyl, is attached to a linear alkyl chain.

The term “aryl” is used herein to refer to an aromatic substituent, which can be a single aromatic ring or multiple aromatic rings that are fused together, linked covalently, or linked to a common group such as a methylene or ethylene moiety. The common linking group can also be a carbonyl as in benzophenone or oxygen as in diphenylether or nitrogen in diphenylamine. The aromatic ring(s) can include phenyl, naphthyl, biphenyl, diphenylether, diphenylamine, and benzophenone among others. In particular embodiments, the term “aryl” means a cyclic aromatic comprising about 5 to about 10 carbon atoms, including 5 and 6-membered hydrocarbon and heterocyclic aromatic rings. As used herein, the term “aryl” also encompasses “heteroaryl” (i.e., aryl groups containing ring atoms other than carbon). Also, the term “aryl” can also included esters and amides related to the underlying aryl group.

An aryl group can be optionally substituted with one or more aryl group substituents which can be the same or different, where “aryl group substituent” includes alkyl, aryl, aralkyl, hydroxy, alkoxyl, aryloxy, aralkoxyl, carboxy, acyl, halo, nitro, alkoxycarbonyl, aryloxycarbonyl, aralkoxycarbonyl, acyloxyl, acylamino, aroylamino, carbamoyl, alkylcarbamoyl, dialkylcarbamoyl, arylthio, alkylthio, alkylene, and —NR′R″, where R′ and R″ can be each independently hydrogen, alkyl, aryl and aralkyl. In this case, the aryl can be referred to as a “substituted aryl”. Specific examples of aryl groups include but are not limited to cyclopentadienyl, phenyl, furan, thiophene, pyrrole, pyran, pyridine, imidazole, isothiazote, isoxazole, pyrazole, pyrazine, pyrimidine, and the like.

The term “alkoxy” is used herein to refer to the —OZ¹ radical, where Z¹ is selected from the group consisting of alkyl, substituted alkyl, cycloalkyl, substituted cycloalkyl, heterocycloalkyl, substituted heterocycloalkyl, silyl groups, and combinations thereof as described herein. Suitable alkoxy radicals include, for example, methoxy, ethoxy, benzyloxy, t-butoxy, etc. A related term is “aryloxy” where Z¹ is selected from the group consisting of aryl, substituted aryl, heteroaryl, substituted heteroaryl, and combinations thereof. Examples of suitable aryloxy radicals include phenoxy, substituted phenoxy, 2-pyridinoxy, 8-quinalinoxy, and the like.

The term “amino” is used herein to refer to the group —NZ¹Z², where each of Z¹ and Z² is independently selected from the group consisting of hydrogen, alkyl, substituted alkyl, cycloalkyl, substituted cycloalkyl, heterocycloalkyl, substituted heterocycloalkyl, aryl, substituted aryl, heteroaryl, substituted heteroaryl, alkoxy, aryloxy, silyl, and combinations thereof. Additionally, the amino group can be represented as —N⁺Z¹Z²Z³, with the previous definitions applying and Z³ being either H or alkyl.

As used herein, the term “acyl” refers to an organic acid group wherein the —OH of the carboxyl group has been replaced with another substituent (i.e., as represented by RCO—, wherein R is an alkyl or an aryl group as defined herein). As such, the term “acyl” specifically includes arylacyl groups, such as an acetylfuran and a phenacyl group. Specific examples of acyl groups include acetyl and benzoyl.

“Aroyl” means an aryl-CO— group wherein aryl is as previously described. Exemplary aroyl groups include benzoyl and 1- and 2-naphthoyl.

“Cyclic” and “cycloalkyl” refer to a non-aromatic mono- or multicyclic ring system of about 3 to about 10 carbon atoms, e.g., 3, 4, 5, 6, 7, 8, 9, or carbon atoms. The cycloalkyl group can be optionally partially unsaturated. The cycloalkyl group also can be optionally substituted with an alkyl group substituent as defined herein, oxo, and/or alkylene. There can be optionally inserted along the cyclic alkyl chain one or more oxygen, sulfur, or substituted or unsubstituted nitrogen atoms, wherein the nitrogen substituent is hydrogen, lower alkyl, or aryl, thus providing a heterocyclic group. Representative monocyclic cycloalkyl rings include cyclopropyl, cyclobutyl, cyclopentyl, cyclohexyl, and cycloheptyl. Multicyclic cycloalkyl rings include adamantyl, octahydronaphthyl, decalin, camphor, camphane, and noradamantyl.

“Aralkyl” refers to an aryl-alkyl- group wherein aryl and alkyl are as previously described. Exemplary aralkyl groups include benzyl, phenylethyl, and naphthylmethy. Similarly, the term “alkylaryl” refers to an alkyl-aryl-group, wherein aryl and alkyl are as previously described. As such, the terms “aralkyl” and “alkylaryl” can be used interchangeably, although in some instances the use of one term versus the other is intended to express the order of a group in a chemical structure when read from left-to-right. By way of example, an “ethylphenyl” substituent might be distinguished from a “phenylethyl” substituent in that in the former case, the ethyl moiety is bound to the main body of the molecule while in the latter it would be the phenyl moiety that is bound to the main body of the molecule.

“Aralkyloxyl” refers to an aralkyl-O— group wherein the aralkyl group is as previously described. An exemplary aralkyloxyl group is benzyloxyl.

“Dialkylamino” refers to an —NRR′ group wherein each of R and R′ is independently an alkyl group as previously described. Exemplary alkylamino groups include ethylmethylamino, dimethylamino, and diethylamino.

“Alkoxycarbonyl” refers to an alkyl-O—CO— group. Exemplary alkoxycarbonyl groups include methoxycarbonyl, ethoxycarbonyl, butyloxycarbonyl, and t-butyloxycarbonyl.

“Aryloxycarbonyl” refers to an aryl-O—CO— group. Exemplary aryloxycarbonyl groups include phenoxy- and naphthoxy-carbonyl.

“Aralkoxycarbonyl” refers to an aralkyl-O—CO— group. An exemplary aralkoxycarbonyl group is benzyloxycarbonyl.

“Carbamoyl” refers to an H₂N—CO— group.

“Alkylcarbamoyl” refers to a R′RN—CO— group wherein one of R and R′ is hydrogen and the other of R and R′ is alkyl as previously described.

“Dialkylcarbamoyl” refers to a R′RN—CO— group wherein each of R and R′ is independently alkyl as previously described.

“Acyloxyl” refers to an acyl-O— group wherein acyl is as previously described.

“Acylamino” refers to an acyl-NH— group wherein acyl is as previously described.

“Aroylamino” refers to an aroyl-NH— group wherein aroyl is as previously described.

The term “amino” refers to the —NH₂ group.

The term “carbonyl” refers to the —(C═O)— group.

The term “carboxyl” refers to the —COOH group.

The terms “halo”, “halide”, or “halogen” as used herein refer to fluoro, chloro, bromo, and iodo groups.

The term “halomethyl” refers to a methyl group wherein at least one hydrogen has been substituted with a halogen.

The term “hydroxyl” refers to the —OH group.

The term “hydroxyalkyl” refers to an alkyl group substituted with an —OH group.

The term “mercapto” refers to the —SH group.

The term “nitro” refers to the —NO₂ group.

The term “oxo” refers to a compound described previously herein wherein a carbon atom is replaced by an oxygen atom.

The term “thio” refers to a compound described previously herein wherein a carbon or oxygen atom is replaced by a sulfur atom.

The term “sulfate” refers to the —SO₄ group.

A “heteroatom”, as used herein, is an atom other than carbon.

Exemplary heteroatoms are heteroatoms selected from the group consisting of N, O, P, S, Si, B, Ge, Sn, and Se. In some embodiments, a heteroatom is N. In some embodiments a heteroatom is O. In some embodiments, a heteroatom is S.

Certain substituents of PM play particular roles in ROS, RCS, and AGE scavenging. For example, the 4-methylamino group is important for scavenging of MGO, and the 4-methylamino group in conjunction with the 3′-hydroxy group are important for scavenging of GO and GLA, for inhibition of post-Amadori oxidative reactions, and for scavenging of hydroxyl radicals. Thus, these positions can also be modified using techniques known to the skilled artisan with the proviso that the substitutions retain some or all of the inhibitory and/or scavenging activities of PM.

IV.D. Formulations

In some embodiments, the methods of the presently disclosed subject matter employ a pharmaceutical composition that includes a pharmaceutically acceptable carrier, such as but not limited to a carrier that is pharmaceutically acceptable in humans. Suitable formulations include aqueous and non-aqueous sterile injection solutions that can contain anti-oxidants, buffers, bacteriostats, bactericidal antibiotics, and solutes that render the formulation isotonic with the bodily fluids of the subject; and aqueous and non-aqueous sterile suspensions, which can include suspending agents and thickening agents. The formulations can be presented in unit-dose or multi-dose containers, for example sealed ampoules and vials, and can be stored in a frozen or freeze-dried (lyophilized) condition requiring only the addition of sterile liquid carrier, for example water for injections, immediately prior to use. Some exemplary ingredients are sodium dodecyl sulfate (SDS), in some embodiments in the range of 0.1 to 10 mg/ml, in some embodiments about 2.0 mg/ml; and/or mannitol or another sugar, in some embodiments in the range of 10 to 100 mg/ml, in some embodiments about 30 mg/ml; and/or phosphate-buffered saline (PBS). Any other agents conventional in the art having regard to the type of formulation in question can be used.

Additionally, PM can be formulated as an intravenous (i.v.) infusion, such as, but not limited to, for use in human subjects. In some embodiments, the i.v. formulation is an aqueous formulation that is pharmaceutically acceptable for use in humans and that comprises a therapeutically effective amount of PM, an analog or derivative thereof, or a pharmaceutically acceptable salt of any of the foregoing, in a buffer that is chosen to maintain the aqueous formulation at a pH of about 7. In some embodiments, the formulation further comprises one or more pharmaceutically acceptable carriers or excipients (e.g., one or more carriers or excipients that are pharmaceutically acceptable for use in humans).

IV.E. Administration

PM administration can be by any method known to one of ordinary skill in the art. In some embodiments, suitable methods for administration of PM include, but are not limited to intravenous administration, bolus injection, and oral administration. In some embodiments, a therapeutically effective amount of PM is administered by initial bolus injection followed by intravenous administration.

IV.F Dose

An effective dose for use in the presently disclosed methods is administered to a subject in need thereof. As used herein, the phrase “effective amount” refers to an amount of a therapeutic composition (e.g., PM, or an analog or derivative thereof, or a pharmaceutically acceptable salt of any of the foregoing, in a pharmaceutically acceptable carrier or excipient) sufficient to produce a biologically or clinically relevant response (e.g., a “benefit”) in a subject being treated. The actual amount delivered can be varied so as to administer an amount that is effective to achieve the desired therapeutic response for a particular subject.

The potency of a composition can vary, and therefore an “effective amount” can vary. However, using standard assay methods, one skilled in the art can readily assess the potency and efficacy of PM, or an analog or derivative thereof, or a pharmaceutically acceptable salt of any of the foregoing, and adjust the therapeutic regimen accordingly.

After review of the disclosure of the presently disclosed subject matter presented herein, one of ordinary skill in the art can tailor the dosages to an individual subject, taking into account the particular formulation, method of administration to be used with the composition, and particular disease process to be treated and/or prevented. Further calculations of dose can consider subject height and weight, severity and stage of symptoms, and the presence of additional deleterious physical conditions. Such adjustments or variations, as well as evaluation of when and how to make such adjustments or variations, are well known to those of ordinary skill in the art of medicine. In some embodiments, the effective amount is selected from the group consisting of less than 1 mg/day, about 1-10 mg/day, about 10-50 mg/day, about 50-100 mg/day, about 100-200 mg/day, about 200-300 mg/day, about 300-400 mg/day, about 400-500 mg/day, and more than 500 mg/day.

As is known in the art, these dosages can be administered at one time or as part of two or more daily administrations. For example, for oral administration, the dose can be in some embodiments about 50 mg/dose bid in die (BID), in some embodiments about 100 mg/dose BID, and in some embodiments about 250 mg/ml BID. For intravenous administration, the daily dose can be in some embodiments about 25 mg/day, in some embodiments about 50 mg/day, and in some embodiments about 200 mg/day. It is understood that the effective amount might vary among patients, and further that the actual dose administered can easily be modified by a physician as needed.

### EXAMPLES

The following Examples provide illustrative embodiments. In light of the present disclosure and the general level of skill in the art, those of skill will appreciate that the following Examples are intended to be exemplary only and that numerous changes, modifications, and alterations can be employed without departing from the scope of the presently disclosed subject matter.

### Example 1

### Pyridoxamine (PM) Inhibits Formation of N^{ε}-carboxymethyllysine

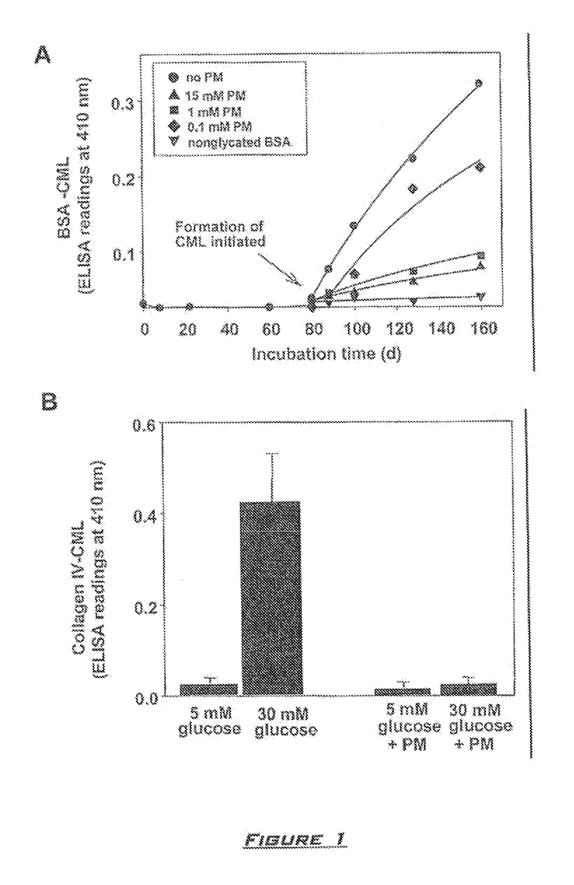

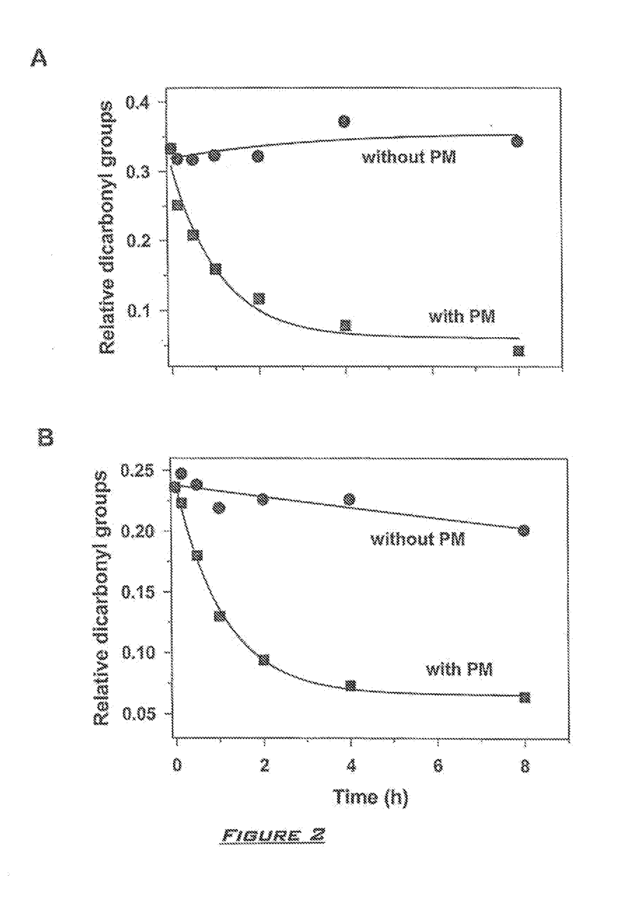

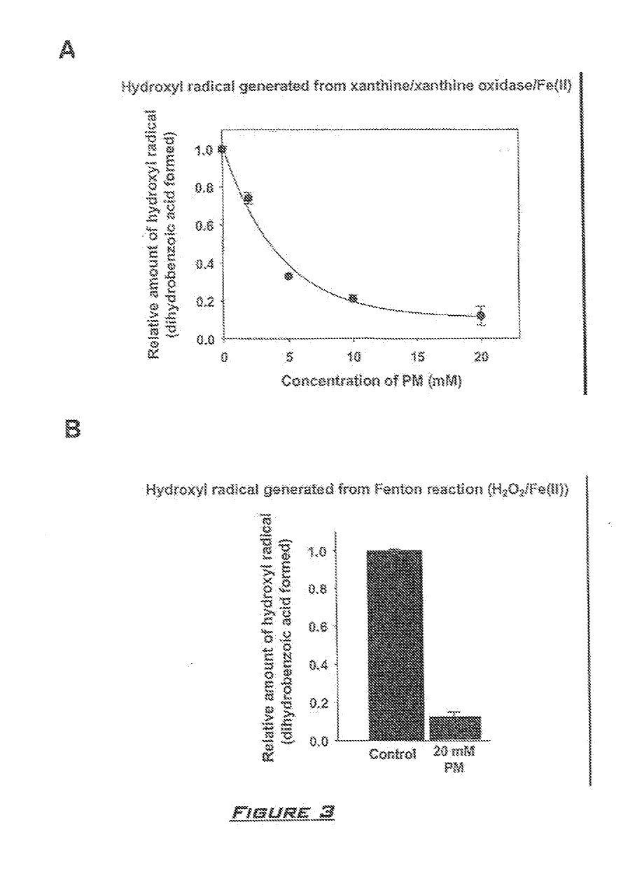

Isolated BSA-Amadori glycation intermediate was formed at diabetic (30 mM) concentration of glucose. The intermediate was incubated in 200 mM sodium phosphate buffer, pH 7.5 at 37° C. with and without PM as indicated in FIG. 1A. The conversion of Amadori intermediate to the AGE N^{ε}-carboxymethyllysine (CML) was measured using an anti-CML antibody. As shown in FIG. 1A, PM inhibited the conversion of the BSA-Amadori glycation intermediate to BSA-CML.

Collagen IV coated on the 96-well plate was incubated in the presence of normal (5 mM) and diabetic (30 mM) concentrations of glucose with or without PM. Incubations were carried out in 200 mM sodium phosphate buffer, pH 7.5 at 37° C. for 40 days. As shown in FIG. 1B, PM inhibited the formation of CML on collagen IV.

### Example 2

### Pyridoxamine Scavenges Reactive Carbonyl Species

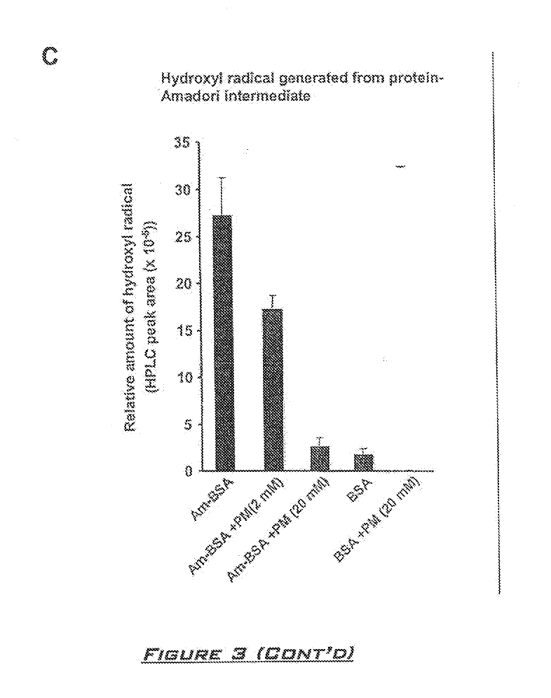

Carbonyl compounds GO and MGO were incubated with or without PM in 200 mM sodium phosphate buffer, pH 7.5 at 37° C. The residual free carbonyl groups were measured using Girard's reagent T. As shown in FIGS. 2A and 2B, PM reduced the levels of dicarbonyl groups for both glyoxal (GO; FIG. 2A) and methylglyoxal (MGO; FIG. 2B).

### Example 3

### Pyridoxamine Inhibits Accumulation of Hydroxyl Radical

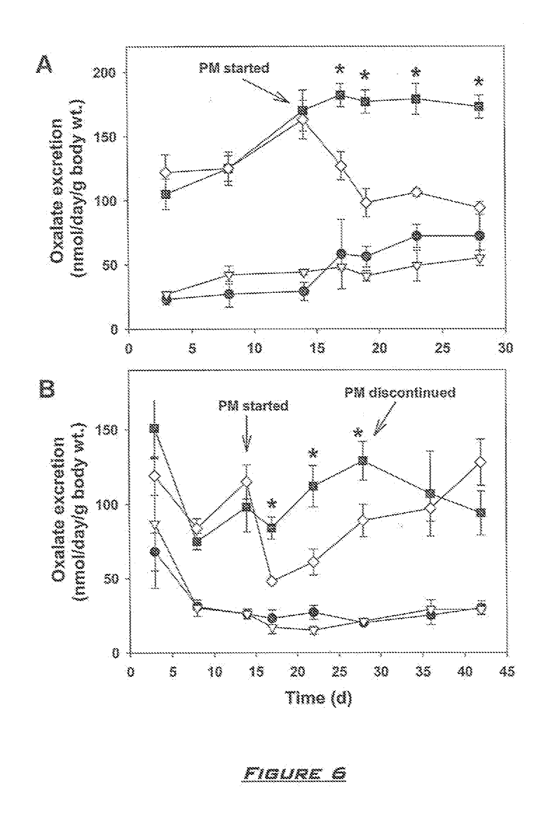

Hydroxyl radical, the most active ROS, was formed using three different reactions: hydroxyl radical generation from xanthine/xanthine oxidase/Fe(II) (see FIG. 3A), hydroxyl radical generation from the Fenton reaction (H₂O₂/Fe(II) (see FIG. 38), and hydroxyl radical generation from a protein-Amadori intermediate (see FIG. 3C). Incubations were carried out with and without PM in 200 mM sodium phosphate buffer, pH 7.5 at 37° C. for 24 hours. Accumulation of hydroxyl radical was measured using specific oxidation of benzoic acid. As shown in FIGS. 3A-3C, the presence of PM inhibited the formation of hydroxyl radicals in each assay.

### Example 4

### PM Protects Renal Cells from Cisplatin-Induced Cell Death

Human glomerular visceral epithelial cells (podocytes) were plated at 8,000 cells/well (96-well plate) and incubated for 24 hours in RPMI medium supplemented with 1% FBS. Following this incubation cells were treated as indicated without changing the medium. The treatment continued for 22 hours and cell viability was measured using Rapid Cell Proliferation Kit (CALBIOCHEM®, a unit of EMD Biosciences. Inc. San Diego, Calif., United States of America).

Cisplatin is a known nephrotoxin capable of inducing oxidative stress and is often used to induce acute renal failure in animal studies (see Bellomo et al. (2004) Group Crit. Care 8:R204-212). As shown in FIG. 4, cisplatin caused a significant decrease in podocyte viability after 22 hours of treatment. Pyridoxamine protected cell viability in a concentration-dependent manner.

### Example 5

### Use of PM to Reduce Oxidative Stress in Kidney in a Rat CCl₄ Model

Based on the results of the in vitro experiments disclosed herein, it was hypothesized that PM might reduce oxidative stress in the kidney in vivo. This hypothesis was evaluated using the carbon tetrachloride rat model of oxidative stress (see Kadiska et at. (2005) Free Radic Biol Med 38:698-710). The effect of PM was determined by measuring the levels of an oxidative stress marker, 8-F₂-isoprostane (8-iso-PGF₂) in the kidney using gas-chromatography/mass-spectrometry (see Morrow et al. (1999) Methods Enzyrmol 300:3-12).

As shown in FIG. 5, PM demonstrated a tendency toward lowering the level of the isoprostane marker. Although the PM effect in this experiment did not reach the level of statistical significance (CCl₄ vs. CCl₄+PM, p=0.066), the tendency was consistent with PM's ability to scavenge hydroxyl radical as well as metal ion catalysts of oxidative reactions. The effect of PM might reach statistical significance if the group size were to be increased to compensate for the known variability of the levels of oxidative stress in CCl₄ model.

The F₂-isoprostanes are lipophilic molecules derived from non-enzymatic oxidation of arachidonic acid. It is also possible that PM might have additional effects on the levels of other markers of oxidative stress such as oxidized proteins.

The loss of epithelial angiotensin I-converting enzyme (ACE) in the proximal tubule and its appearance in urine is characteristic for different stages of acute renal failure (Metzger at al. (1999) Kidney Intl 56:1442-1454). The increase in urinary ACE has been demonstrated in several models of acute renal failure (Pedraza-Chaverri et al. (1995) Ren Fail 17:365-375; Pedraza-Chaverri et al. (1995) Ren Fail 17:377-388). Similar increase in urinary ACE has been reported in CCl₄ rat model, suggesting that damage to proximal tubule epithelium, a characteristic feature of acute renal failure, also occurs in this model (Pedraza-Chaverri et al. (1993) Ren Fail 15:19-26).

### Example 6

### Ethylene Glycol Models of Hyperoxaluria-Induced ARF

Reagents

Ethylene glycol, glycolic acid, glycolate oxidase, glyoxylic acid, glycolaldehyde, trinitrobenzenesulfonic acid were purchased from Sigma-Aldrich Co. (St. Louis, Mo., United States of America). Pyridoxamine was generously provided by BioStratum, Inc. (Durham, N.C., United States of America).

### Ethylene Glycol Model, of Hyperoxaluria and Pyridoxamine Treatment.

An established rat model of experimental hyperoxaluria, the ethylene glycol (EG) model (see e.g., Lyon et al. (1996) Invest Urol 4:143-151; Khan (1997) World J Urol 05:236-243) was employed. Although rats do not spontaneously develop stones, hyperoxaluria can be induced in rats, and, as in humans, their oxalate synthesis occurs primarily via glyoxylate pathway (Khan (1997) World J Urol 05:236-243). Because ethylene glycol is converted to glycolaldehyde, an intermediate in glyoxylate pathway, its administration results in increased urinary oxalate levels (Lyon et al. (1996) Invest Urol 4:143-151; Khan (1997) World J Urol 05:236-243).

Animal experiments were performed at the Association for Assessment and Accreditation of Laboratory Animal Care International-accredited animal facilities at Vanderbilt University Medical Center (Nashville, Tenn., United States of America) and University of Kansas Medical Center (Kansas City, Kans., United States of America) according to institutional guidelines and Institutional Animal Care and Use Committee-approved experimental protocols. Sprague-Dawley male rats (49-52 days old; Harlan Bioproducts, Inc., Indianapolis, Ind., United States of America) were housed individually and fed standard powdered stock ration (Purina Mill Inc., St. Louis, Mo., United States of America). The temperature was kept at 22±2° C. with the lights set at a 12-hour light/dark cycle.

For uniform administration of EG and PM, water supply to all animals was limited to 45 ml/day. PM was given to animals in drinking water after a two-week adaptation period to establish elevated constant levels of urinary oxalate excretion in model animals. To minimize possible chemical degradation of PM, a light sensitive compound, fresh solution was prepared daily and administered in water bottles wrapped in aluminum foil. The length of treatment was determined based on the fact that after about 35 days of experimental hyperoxaluria rats may have some evidence of microscopic nephrolithiasis, but their renal function remains normal (Khan (1997) World J Urol 05:236-243).

Sprague-Dawley male rats (49-52 days old) were randomized on Day 1 to receive either EG (0.75% v/v in drinking water; EG group) or water (Control group). After Day 14 animals within each group (Control, solid circles, or EG, solid squares, in FIG. 6) were pair-matched according to their urinary oxalate level. One member of each pair was then randomly assigned to receive PM (3 mg/ml) either in drinking water (PM group) or in 0.75% ethylene glycol (EG+PM group). 24-hour urine samples were collected in metabolic cages under toluene (to inhibit bacteria growth) in 50 ml tubes with hydrochloric acid to minimize spontaneous breakdown of urinary ascorbic acid to oxalate. The samples were analyzed immediately or stored at −70° C. until further analysis.

Analysis of Urine Samples.

Urinary oxalate was measured by the oxalate oxidase method. Briefly, the method is based on the conversion of oxalate to hydrogen peroxide and carbon dioxide by oxalate oxidase. The latter is then determined enzymatically with horseradish peroxidase by oxidative coupling of 3-methyl-2-benzothiazolinone hydrazone with N,N-dimethylaniline. The resulting colored product is determined spectrophotometrically at 595 nm (Laker at al. (1980) Clin Chem 26:827-830). Urinary calcium was measured using the Calcium Assay Kit (Diagnostic Chemical Ltd., Charlottetown, Canada). Urinary creatinine was determined using the Creatinine Kit (Sigma Co., St. Louis, Mo., United States of America) based on the Jaffe colorimetric assay, with modifications to improve specificity.

Urinary concentration of glycolic acid was determined by the method described by Petrarulo et al. ((1989) J Chromatogr 465:87-93). The method is based on enzymatic conversion of glycolic acid to glyoxylic acid followed by derivatization with phenylhydrazone, separation of reaction products by reverse phase HPLC (NovaPack C18 column, Waters Corp., Milford, Mass., United States of America), and spectrophotometric detection at 324 nm. Since high concentrations of PM can be present in urine of PM treated animals, whether this would interfere with the analysis of urine samples was investigated. The addition of PM to the assay mixtures (final concentration of 3 mg/ml) did not affect the readings.

Effect of Pyridoxamine Treatment on Urinary Oxalate Excretion.

The dose of PM for animal treatment (180 mg/day/kg body wt.) was chosen based on the results of long-term animal studies in a diabetic rat model, where similar or higher PM doses were safe and had therapeutic effects (Degenhardt et al. (2002) Kidney Intl 61:939-950; Stitt et al. (2002) Diabetes 51:2826-2832). Animals in all experimental groups showed no adverse effects. No significant differences in weight gain were detected for the duration of the experiment. Animals with experimental hyperoxaluria (EG group) exhibited about a 4-fold increase in urinary oxalate excretion, consistent with previously published data (Khan (1997) World J Urol 05:236-243). PM treatment of these animals (EG+PM group) resulted in a dramatic and sustained decrease in urinary oxalate excretion (see FIG. 6A). In a separate experiment, PM was discontinued after a significant reduction in oxalate excretion was achieved (see FIG. 6B). This resulted in an increase in urinary oxalate excretion to the levels found in untreated hyperoxaluric animals, confirming that the observed effect is dependent on the PM treatment (FIG. 6B). With the proposed experimental design, interference between PM and ethylene glycol might have been possible because both were administered in drinking water. If significant spontaneous oxidation of ethylene glycol occurs in solution, the resulting carbonyl moieties might react with the PM amino group. To address this question, the amount of reactive PM amino groups was measured in incubations with ethylene glycol using TNBS assay (Spadaro et al. (1979) Anal Biochem 96:317-321). No change in amino groups was detected after 24 hours at room temperature.

### Example 7

### Use of PM to Inhibit Oxidative Stress-Induced Inflammation In Vivo

The ability of PM to inhibit oxidative stress-induced inflammation is tested in vivo. Oxidative stress is induced in animals (e.g., in mice), and the ability of PM to inhibit the inflammatory response to oxidative-stress is tested. Before and after the induction of oxidative stress, various markers of the inflammatory response are assayed, including but not limited to prostaglandins, cyclooxygenase-2, interleukins (e.g., IL-6, IL-8), tumor necrosis factors (e.g., TNF-α), and cell adhesion molecules. Also assayed is the production of AGEs, ROS, and RCS.

### Example 8

### Treatment of Gentamicin and Glycerol Models of ARF with PM

As disclosed herein. PM can inhibit generation and/or accumulation of ROS in vitro and can inhibit oxidative stress when administered in vivo to test subjects. In the instant Example, whether PM has therapeutic effect in animal models of ARF characterized by increased oxidative stress is examined.

The Gentamicin and the Glycerol Rat Models of ARF.

There are several animal models of ARF, each having its specific advantages and limitations (see e.g., Bellomo et al. (2004) Group Crit. Care 8:R204-12). Two such models are the gentamicin and the glycerol models. The relationship between oxidative stress, in particular generation of hydroxyl radical, and ARF has been previously demonstrated using these models (Baliga et al. (1999) Drug Metab Rev 31:971-97). The protective effects of hydroxyl radical scavengers and/or iron chelators have also been demonstrated in these models, even though no successful therapies have been subsequently developed as a result of these findings. Importantly, the gentamicin and the glycerol models are highly relevant, representing two of the major clinical causes of ARF: antibiotic toxicity and rhabdomyolosis, respectively (Bellomo et at. (2004) Group Crit. Care 8:R204-12). Examining the effects of PM in two different models also helps to minimize possible model-specific adverse effects.

The primary outcome measured is serum creatinine level at the end of PM treatment. An important secondary outcome is pathologic changes in the tubulointerstitial areas of the kidney observed in the PAS-stained kidney sections at the end of PM treatment. It is noted that the accuracy of creatinine clearance measurement in ARF can be limited due to an increase in tubular creatinine secretion and the tubular back-leak and that serum creatinine concentration alone can provide an inaccurate estimation of GFR under the non-steady-state condition of ARF. However, the change of serum creatinine level from the baseline reflects the change in GFR (Bellomo et al. (2004) Group Crit. Care 8:R204-12).

Model Optimization.

The course of ARF in animal models can vary depending on specific experimental conditions. It is also beneficial to assess the effects of the treatment before ARF enters the recovery phase. Therefore, the time course of ARF in the gentamicin and the glycerol models is characterized under the disclosed experimental conditions.

Two-month-old male Sprague-Dawley rats (250-300 g) are housed individually in metabolic cages. The temperature is kept at 22±2° C. and the lights are set at a 12-hour light/dark cycle. Animals are fed standard powdered stock ration, and their body weights are monitored daily. After a 2-day adaptation period, blood samples are drawn from the tail vein for the baseline serum creatinine measurements.

To characterize the gentamicin model under the disclosed experimental conditions, animals are randomized based on serum creatinine levels into 6 groups. Five groups receive subcutaneous injections of gentamicin (400 mg/day/kg body wt, divided into several doses) every 8 hours for 2 days (see e.g., Goto et al. (2004) Virchows Arch 444:362-74). Animals are euthanized (sodium pentobarbital, 150 mg/kg IP) either immediately after the last injection (Day 0) or at Day 2, 4, 6, or 10 after the last injection. The control group receives injections of isotonic sterile saline and is euthanized at Day 10 after the last injection. Serum creatinine levels and renal pathology are determined as described hereinbelow. The time after gentamicin treatment when maximum increase in serum creatinine level is achieved is determined and used in additional experiments with PM. The predicted 2.5-fold maximum rise in serum creatinine levels in gentamicin-treated rats compared to that in controls is based on previous reports (see e.g., Pedraza-Chaverri et al. (2003) Eur J Pharmacol 473:71-8; Goto et at. (2004) Virchows Arch 444:362-74).

Computations for the sample-size determination are made using a PS computer program, version 2.1.31 (see Dupont & Plummer, Jr. (1990) Control Clin Trials 11:116-28). The sample size required to achieve 90% power with a two-tailed t-test is found to be 3 animals per group. To account for possible data loss, a sample size of 4 or more animals per group is employed.

To characterize the glycerol model under the experimental conditions disclosed herein, rats are randomized based on their serum creatinine levels into 5 groups (n=4). Animals are dehydrated for 24 hours and injected under gas phase vapor anesthesia with 50% glycerol solution (8 ml/kg body wt, one-half dose in each hindlimb muscle). After the injection, animals are allowed free excess to water (Shah et al. (1988) Am J Physiol 255:F438-43). Rats are euthanized at 0, 6, 24, and 48 hours after glycerol injection. The control group receives saline injections and euthanized after 48 hours. Serum creatinine levels and renal pathology are determined as described hereinbelow. As with the gentamicin model, the time when maximum rise in serum creatinine level is achieved is used in subsequent experiments with PM. The development of ARF in both models is also confirmed by the analysis of kidney sections for pathogenic changes.

Treatment of the Animal Models with PM.

Because ARF develops very rapidly, effective treatment might not be always possible, especially after ARF progresses to the maintenance phase (Molitoris (2003) J Am Soc Nephrol 14:265-7). Therefore, in situations when the predicted occurrence of ARF is high, such as in critical care, prophylactic therapy can be preferable. Both prophylactic and therapeutic treatments with PM are under the controlled conditions provided by animal models of ARF.

For PM experiments, rats are randomized into 4 groups based on their baseline serum creatinine levels: (a) the Control group (no ARF is induced); (b) the PM group (no ARF is induced, but PM is given in drinking water); (c) the ARF group; and (d) the ARF+PM group. The experiment is terminated when the serum creatinine in the ARF group reaches its maximum level as determined in the model optimization studies described hereinabove. At this time point, serum creatinine is measured and kidney pathology evaluated in all groups as described hereinbelow. The dose of PM is 180 mg/day/kg body weight. This PM dose was shown in previous studies to be well tolerated and effective (see Chetyrkin et el. (2005) Kidney Intl 67:53-60) and also was used in the Examples described herein. To ensure a more uniform administration of PM, water supply is limited to 45 ml/day, an average daily water consumption by rats. Such water limitation does not cause any noticeable discomfort to animals (see Chetyrkin et al. (2005) Kidney Intl 67:53-60). To minimize possible chemical degradation of PM, which is a light-sensitive compound, fresh solutions are prepared daily and administered in water bottles wrapped in aluminum foil.

For prophylactic treatment testing, PM is given to animals 24 hours prior to the Induction of ARF in either the gentamicin or the glycerol models. The PM treatment is continued until the end of experiment. For therapeutic treatment testing, PM is given immediately after the induction of ARF and continued until the end of experiment.

Creatinine levels at the end of experiment (the primary outcome) are envisioned to be significantly lower in the ARF+PM group compared to that in the ARF group. The degree of renal tubular pathology at the end of experiment (the secondary outcome) is also envisioned to be lower in the ARF+PM group compared to the ARF group. The sample size to achieve 90% power with a two-tailed t-test is envisioned to be 8 animals per group. To account for possible data loss, a sample size of 9 or more animals per group is employed.

Sample Analysis.

Rat blood is collected from the tail vein or, after euthanasia, from the portal vein. Serum is prepared and stored at −70° C. Serum creatinine is measured using the Creatinine Kit (Diagnostic Chemicals Ltd., Charlottetown, Canada) based on the Jaffe colorimetric assay.

After euthanasia, kidneys are removed. For each kidney, two PAS-stained paraffin sections are prepared on a slide and slides are coded for blind scoring. The morphologic changes in renal tubulointerstitial area are scored using the technique of Houghton at al. (1978) Am J Pathol 93:137-52. Scores represent the degree of epithelial cell disintegration and tubular epithelial necrosis, and the presence of granular debris in tubular lumen (Houghton et al. (1978) Am J Pathol 93:137-52).

Statistical analyses are performed using post-hoc Student-Newman-Keuls comparisons. P≦0.05 is taken to signify statistical significance.

### Example 9

### PM Treatment and Oxidative Stress in Animal Models of ARF

In this Example, whether PM treatment can lower the levels of different markers of oxidative stress in ARF animal models is assessed.

Plasma and kidneys are collected as in the Examples described hereinabove. The kidney tissue designated for the isoprostane measurements is flash frozen in liquid nitrogen and kidney and plasma samples are stored at −70° C. until analysis. The degree of oxidative stress and the PM effects on oxidative stress is determined by quantitative measurements of several markers: plasma protein thiols, protein-associated carbonyl groups, cytokine TNF-α, and 8-iso-PGF₂ isoprostane. The levels of protein thiols, protein carbonyl groups, and TNF-α have been shown to be specifically affected in critical care patients with ARF (see Himmelfarb et al. (2004) J Am Soc Nephrol 15:2449-56).

Plasma protein thiol oxidation is assayed according to the modified Ellman method using 5′5′-dithio-bis[2-nitrobenzoic acid](DTNB) reagent as described in Hu at al. (1993) J Lab Clin Med 121:257-62. Sample absorbance is read at 412 nm on a SPECTRAMAX® 190 spectrophotometer (Molecular Devices Corp., Sunnyvale, Calif., United States of America). The concentration of thiol groups is calculated using the TNB molar extinction coefficient of 14,100 M/cm.

Protein-associated carbonyl groups are measured using the Protein Carbonyl Enzyme Immunoassay Kit (Zenith Technologies Ltd., Dunedin, New Zealand). This kit uses derivatization of protein carbonyls in samples and oxidized protein standards with dinitrophenylhydrazine (DNP), followed by ELISA with an anti-DNP antibody.

Plasma TNF-α concentration is determined by ELISA using a kit from BioSource International (Camarillo, Calif., United States of America).

Analysis of 8-iso-PGF₂ in plasma and kidney tissue is performed by GC/MS following lipid extraction and the HPLC purification as described in Morrow et al. (1999) Methods Enzymol 300:3-12.

### Example 10

### Prevention of ARF in Critically Ill Patients

The safety and effectiveness of pyridoxamine treatment in reducing the Incidence of acute renal failure in critically ill patients is tested. The duration of most patients in the ICU does not exceed two months, and therefore the treatment period to prevent a critically ill patient from developing ARF is unlikely to be much longer than two months. The definition of primary and secondary endpoints and patient recruitment criteria are made in accordance with recognized parameters in the art. Molecular urinalysis and the analysis of established biomarkers of oxidative stress and carbonyl stress are also employed before, during, and after the completion of therapy. See e.g., U.S. Pat. No. 6,953,666, incorporated herein by reference in its entirety.

### Example 11

### Treatment of ARF in Critically Ill Patients

The safety and effectiveness of pyridoxamine treatment in reducing the incidence of mortality in critically ill patients that develop ARF is evaluated, A number of studies indicate the mortality rate of ARF in the ICU is 50 to 70 percent. Recruitment criteria are developed in accordance with recognized parameters in the art. Molecular urinalysis and the analysis of established biomarkers of oxidative stress and carbonyl stress are also performed before, during, and after the completion of therapy.

### Example 12

### Clinical Trial Design

A randomized, placebo-controlled, double blind study is conducted. Critically ill patients with established acute renal failure are recruited based on art-recognized parameters and published criteria of acute kidney injury. The primary end-point is 28-day mortality (in hospital and intensive care unit stay). Certain clinically relevant outcomes, including but not limited to renal recovery rates, ICU stay and days on ventilator, are also determined.

### REFERENCES

The references listed below, as well as all references cited in the specification, are incorporated herein by reference to the extent that they supplement, explain, provide a background for, and/or teach methodology, techniques, and/or compositions employed herein.
Bellomo et al. (2004) Group Crit. Care 8:R204-12.
Baliga at al. (1999) Drug Metab Rev 31:971-97.
Chetyrkin et al. (2005) Kidney Intl 67:53-60.
Dupont & Plummer, Jr. (1990) Control Clin Trials 11:116-28.
Goto et al. (2004) Virchows Arch 444:362-74.
Himmelfarb et al. (2004) J Am Soc Nephrol 15:2449-56.
Houghton et al. (1978) Am J Pathol 93:137-52.
Hu et al. (1993) J Lab Clin Med 121:257-62.
Kadliska et al. (2005) Free Radic Biol Med 38:698-710.
Molitoris (2003) J Am Soc Nephrol 14:265-7.
Morrow et al. (1999) Methods Enzymol 300:3-12.
Pedraza-Chaverri et al. (2003) Eur J Pharmacol 473:71-8.
Shah et al. (1988) Am J Physiol 255:F438-43.
U.S. Pat. Nos. 5,985,857; 6,472,400; 6,716,858; 6,730,686; 6,740,668; and 6,750,209.
Voziyan and Hudson (2005) Ann N Y Acad Sci 1043:807-16.
Voziyan and Hudson (2005) Cell Mol Life Sci 62:1671-81.

It will be understood that various details of the presently disclosed subject matter can be changed without departing from the scope of the presently disclosed subject matter. Furthermore, the foregoing description is for the purpose of illustration only, and not for the purpose of limitation.

We claim:

1. A method for ameliorating at least one symptom of a kidney disorder associated with oxidative stress, carbonyl stress, or combinations thereof in a subject, the method comprising administering to the subject an effective amount of pyridoxamine (PM), or an analog or derivative thereof, or a pharmaceutically acceptable salt of any of the foregoing, whereby at least one symptom of the kidney disorder associated with oxidative stress, carbonyl stress, or combinations thereof in a subject is ameliorated.

2. The method of claim 1, wherein the kidney disorder associated with oxidative stress, carbonyl stress, or combinations thereof in the subject results from a medical condition associated with elevated levels of reactive carbonyl species (RCS), reactive oxygen species (ROS), advanced glycation end products (AGE), or combinations thereof.

3. The method of claim 2, wherein the administering reduces formation of, reactivity of, or both formation of and reactivity of at least one RCS, ROS, of AGE.

4. The method of claim 1, wherein the kidney disorder associated with oxidative stress, carbonyl stress, or combinations thereof in the subject is selected from the group consisting of diabetic nephropathy, acute renal injury, acute renal failure, and combinations thereof.

5. The method of claim 4, wherein the kidney disorder associated with oxidative stress, carbonyl stress, or combinations thereof in the subject is selected from the group consisting of acute kidney injury and acute kidney failure.

6. A method for treating or preventing a nephropathy in a subject, the method comprising administering to the subject an effective amount of pyridoxamine, or an analog or derivative thereof, or a pharmaceutically acceptable salt of any of the foregoing, wherein the effective amount comprises an amount sufficient to ameliorate the nephropathy in the subject, to prevent or delay onset of the nephropathy in the subject, or combinations thereof.

7. The method of claim 6, wherein the nephropathy is selected from the group consisting of acute kidney injury and acute kidney failure.

8. The method of claim 7, wherein the nephropathy is associated with exposure to a radiographic contrast agent, sepsis, chemotherapy, congestive heart failure, cardiovascular disease, or combinations thereof.

9. A method for treating or preventing acute renal injury, acute renal failure, or combinations thereof in a subject, the method comprising administering to the subject an effective amount of pyridoxamine, or an analog or derivative thereof, or a pharmaceutically acceptable salt of any of the foregoing.

10. The method of claim 9, wherein the acute renal injury, acute renal failure, or combinations thereof is associated with exposure to a radiographic contrast agent, sepsis, chemotherapy, congestive heart failure, cardiovascular disease, or combinations thereof.

11. The method of claim 9, wherein the amount administered to the subject is an amount sufficient to increase the time required for at least one symptom associated with the acute renal failure to develop, to reduce the severity of at least one symptom associated with the acute renal failure, to reduce the time that at least one symptom associated with the acute renal failure is present within the subject, and combinations thereof.

12. The method of claim 1, wherein the administering is by a route selected from the group consisting of intravenous administration, parenteral administration, and oral administration.

13. The method of claim 1, wherein the effective amount is selected from the group consisting of less than about 1 mg/day, about 1-10 mg/day, about 10-50 mg/day, about 50-100 mg/day, about 100-200 mg/day, about 200-300 mg/day, about 300-400 mg/day, about 400-500 mg/day, and more than 500 mg/day.

14. A formulation comprising pyridoxamine, or an analog or derivative thereof, or a pharmaceutically acceptable salt of any of the foregoing, wherein the formulation is adapted for intravenous administration into a subject.

15. The formulation of claim 14, wherein the formulation is adapted for intravenous administration into a human.

16. The formulation of claim 15, wherein the formulation comprises an amount of pyridoxamine, or the analog or derivative thereof, or the pharmaceutically acceptable salt of any of the foregoing, sufficient to ameliorate at least one symptom associated with acute renal failure in the subject, to prevent or delay onset of acute renal failure in the subject, or combinations thereof.
